(19) United States
(12) Patent Application Publication
(10) Pub. No.: US 2025/0359741 A1
(43) Pub. Date: Nov. 27, 2025

(54) MEDICAL DEVICE, MEDICAL SYSTEM, MEDICAL DEVICE OPERATION METHOD, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Maho NISHIYAMA, Tokyo (JP)

(73) Assignee: OLYMPUS MEDICAL SYSTEMS CORP., Tokyo (JP)

(21) Appl. No.: 19/291,019

(22) Filed: Aug. 5, 2025

Related U.S. Application Data
(63) Continuation of application No. PCT/JP2023/004453, filed on Feb. 9, 2023.

(51) Int. Cl. A61B 1/04; A61B 1/00; A61B 18/00; A61B 18/04

(52) U.S. Cl. CPC A61B 1/043; A61B 1/000094; A61B 1/00045; A61B 1/00165; A61B 18/04; A61B 2018/00595

(57) ABSTRACT

A medical device includes: a processor including hardware, the processor being configured to generate a fluorescence image based on fluorescence generated by excitation light that excites a substance produced by cauterization using an energy device, determine, based on output information on the energy device and on the fluorescence image, an off-time generated fluorescent region that has been generated during an off-state of output of the energy device, and when it is determined to be the off-time generated fluorescent region, execute a notification process of notifying that a fluorescent region has been generated during the off-state of output of the energy device.

### CROSS REFERENCES TO RELATED APPLICATIONS

This application is a continuation of International Application No. PCT/JP2023/004453, filed on Feb. 9, 2023, the entire contents of which are incorporated herein by reference.

### BACKGROUND

### 1. Technical Field

The present disclosure relates to a medical device, a medical system, a medical device operation method, and a computer-readable recording medium, which are for image processing and outputting an imaging signal resulting from imaging of a subject.

### 2. Related Art

In the related art, a surgical endoscope is inserted in a subject and an operating surgeon cauterizes and treats biological tissue by means of a treatment tool, such as an energy device, while observing a region to be treated (see, for example, International Publication Pamphlet No. WO 2020/174666).

When the biological tissue is cauterized, advanced glycation end-products (AGEs), so-called “burns”, are produced as a result of thermal denaturation. Light of specific wavelengths causes these AGEs to emit fluorescence. The operating surgeon is able to check a region of the thermal denaturation in the region to be treated by observing an image of the fluorescence emitted by the AGEs.

### SUMMARY

In some embodiments, a medical device includes: a processor including hardware, the processor being configured to generate a fluorescence image based on fluorescence generated by excitation light that excites a substance produced by cauterization using an energy device, determine, based on output information on the energy device and on the fluorescence image, an off-time generated fluorescent region that has been generated during an off-state of output of the energy device, and when it is determined to be the off-time generated fluorescent region, execute a notification process of notifying that a fluorescent region has been generated during the off-state of output of the energy device.

In some embodiments, a medical device includes: a processor comprising hardware, the processor being configured to determine, based on a fluorescence image based on fluorescence generated by excitation light that excites a substance produced by cauterization using an energy device and output information on the energy device, an off-time generated fluorescent region that has been generated during an off-state of output of the energy device, and when it is determined to be the off-time generated fluorescent region, execute a notification process of notifying that a fluorescent region has been generated during the off-state of output of the energy device.

In some embodiments, a medical system includes: an imaging device configured to image a subject; a light source configured to emit excitation light that excites a substance produced by a heat treatment on a biological tissue; and a control device that the imaging device is attachable to and detachable from, the control device including a processor and being capable of communicating with a controller that controls an energy device configured to cauterize a treatment target, the processor being configured to generate a fluorescence image based on fluorescence generated by the excitation light that excites the substance produced by cauterization using the energy device, determine, based on output information on the energy device and on the fluorescence image, an off-time generated fluorescent region that has been generated during an off-state of output of the energy device, and when it is determined to be the off-time generated fluorescent region, execute a notification process of notifying that a fluorescent region has been generated during the off-state of output of the energy device.

In some embodiments, provided is a medical device operation method executed by a medical device. The method includes: generating a fluorescence image based on fluorescence generated by excitation light that excites a substance produced by cauterization using an energy device, determining, based on output information on the energy device and on the fluorescence image, an off-time generated fluorescent region that has been generated during an off-state of output of the energy device, and when it is determined to be the off-time generated fluorescent region, notifying that a fluorescent region has been generated during the off-state of output of the energy device.

In some embodiments, provided is a non-transitory computer-readable recording medium with an executable program stored thereon. The program causes a medical device to execute: generating a fluorescence image based on fluorescence generated by excitation light that excites a substance produced by cauterization using an energy device, determining, based on output information on the energy device and on the fluorescence image, an off-time generated fluorescent region that has been generated during an off-state of output of the energy device, and when it is determined to be the off-time generated fluorescent region, notifying that a fluorescent region has been generated during the off-state of output of the energy device.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope system according to a first embodiment;

FIG. 2 is a diagram illustrating a schematic configuration of a treatment system to be connected to the endoscope system according to the first embodiment;

FIG. 3 is a block diagram illustrating a functional configuration of main parts of the endoscope system according to the first embodiment;

FIG. 4 is a diagram schematically illustrating wavelength characteristics of light emitted by first and second light source units, according to the first embodiment;

FIG. 5 is a diagram schematically illustrating a configuration of a pixel unit according to the first embodiment;

FIG. 6 is a diagram schematically illustrating a configuration of a color filter according to the first embodiment;

FIG. 7 is a diagram schematically illustrating sensitivity characteristics of each filter;

FIG. 8A is a diagram schematically illustrating signal values of G pixels of an imaging element according to the first embodiment;

FIG. 8B is a diagram schematically illustrating signal values of R pixels of the imaging element according to the first embodiment;

FIG. 8C is a diagram schematically illustrating signal values of B pixels of the imaging element according to the first embodiment;

FIG. 9 is a diagram schematically illustrating a configuration of a cut filter according to the first embodiment;

FIG. 10 is a diagram schematically illustrating transmission characteristics of the cut filter according to the first embodiment;

FIG. 11 is a diagram schematically illustrating observation principles for a normal light observation mode according to the first embodiment;

FIG. 12 is a diagram schematically illustrating observation principles for a heat treatment observation mode according to the first embodiment;

FIG. 13 is a flowchart illustrating a thermal denaturation region determination process using the endoscope system according to the first embodiment;

FIG. 14 is a diagram for description of a fluorescent image in a fluorescence observation mode;

FIG. 15 is a flowchart illustrating a thermal denaturation region determination process using an endoscope system according to a modified example of the first embodiment;

FIG. 16 is a diagram illustrating a schematic configuration of an endoscope system according to a second embodiment;

FIG. 17 is a block diagram illustrating a functional configuration of main parts of the endoscope system according to the second embodiment; and

FIG. 18 is a diagram illustrating a schematic configuration of a surgical microscope system according to a third embodiment.

### DETAILED DESCRIPTION

Embodiments of the present disclosure will hereinafter be described in detail, together with the drawings. The present disclosure is not to be limited by the following embodiments. The drawings referred to in the following description merely illustrate shapes, sizes, and positional relations schematically to enable the present disclosure to be understood. That is, the present disclosure is not to be limited to just the shapes, sizes, and positional relations exemplified by the drawings. Like portions will be assigned with like reference signs throughout the description and the drawings. An endoscope system including a rigid scope and a medical imaging device will be described as an example of an endoscope system according to the present disclosure.

### First Embodiment

### Configuration of Endoscope System

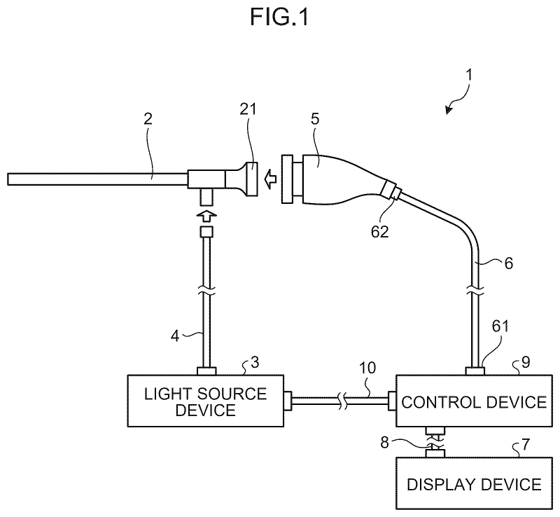

FIG. 1 is a diagram illustrating a schematic configuration of an endoscope system according to a first embodiment. An endoscope system 1 illustrated in FIG. 1 is a system that is used in the medical field, the system being for observation of biological tissue in a subject, such as a living body. The endoscope system 1 is used when a subject is operated or treated using a treatment tool (not illustrated in the drawings), such as an energy device enabling heat treatment. An operating surgeon performs an operation or treatment while observing a display device having an observation image displayed thereon, the observation image being based on image data resulting from imaging by means of a medical imaging device.

The endoscope system 1 includes an insertion unit 2, a light source device 3, a light guide 4, an endoscope camera head 5 (medical imaging device), a first transmission cable 6, a display device 7, a second transmission cable 8, a control device 9, and a third transmission cable 10.

The insertion unit 2 is rigid, or at least part of the insertion unit 2 is flexible, the insertion unit 2 having an elongated shape. The insertion unit 2 is inserted in a subject, such as a patient, via a trocar. The insertion unit 2 has an optical system provided therein, the optical system being, for example, a lens to form an observation image.

One end of the light guide 4 is connected to the light source device 3; and under control by the control device 9, the light source device 3 supplies, to the one end of the light guide 4, illumination light to be emitted to the interior of the subject. The light source device 3 is implemented using: one or more light sources selected from a group consisting of a light emitting diode (LED) light source, a xenon lamp, and a semiconductor laser element, such as a laser diode (LD); a processor that is a processing device having hardware, such as a field programmable gate array (FPGA) or a central processing unit (CPU); and a memory that is a temporary storage area used by the processor.

The one end of the light guide 4 is detachably connected to the light source device 3, and the other end of the light guide 4 is detachably connected to the insertion unit 2. The light guide 4 guides the illumination light supplied from the light source device 3 from the one end to the other end to supply the illumination light to the insertion unit 2.

An eyepiece unit 21 of the insertion unit 2 is detachably connected to the endoscope camera head 5. Under control by the control device 9, the endoscope camera head 5 optically receives the observation image formed by the insertion unit 2, photoelectrically converts the observation image into an imaging signal (RAW data), and outputs this imaging signal to the control device 9 via the first transmission cable 6.

One end of the first transmission cable 6 is detachably connected to the control device 9 via a video connector 61, and the other end of the first transmission cable 6 is detachably connected to the endoscope camera head 5 via a camera head connector 62. The first transmission cable 6 transmits the imaging signal output from the endoscope camera head 5 to the control device 9 and transmits set data and electric power, for example, output from the control device 9 to the endoscope camera head 5. The set data include a control signal, a synchronization signal, and a clock signal, for example, for controlling the endoscope camera head 5.

Under control by the control device 9, the display device 7 displays: an observation image based on an imaging signal that has been subjected to image processing by the control device 9; and various kinds of information related to the endoscope system 1. The display device 7 is implemented using a display monitor of, for example, liquid crystal or organic electroluminescence (EL).

One end of the second transmission cable 8 is detachably connected to the display device 7, and the other end of the second transmission cable 8 is detachably connected to the control device 9. The second transmission cable 8 transmits the imaging signal that has been subjected to the image processing by the control device 9, to the display device 7.

The control device 9 is implemented using: a processor that is a processing device having hardware, such as a graphics processing unit (GPU), an FPGA, or a CPU; and a memory that is a temporary storage area used by the processor. According to a program recorded in the memory, the control device 9 integrally controls operation of the light source device 3, the endoscope camera head 5, and the display device 7 via the first transmission cable 6, the second transmission cable 8, and the third transmission cable 10. Furthermore, the control device 9 executes various kinds of image processing of the imaging signal input via the first transmission cable 6 and outputs this processed imaging signal to the second transmission cable 8.

One end of the third transmission cable 10 is detachably connected to the light source device 3, and the other end of the third transmission cable 10 is detachably connected to the control device 9. The third transmission cable 10 transmits control data from the control device 9 to the light source device 3.

### Functional Configuration of Treatment System

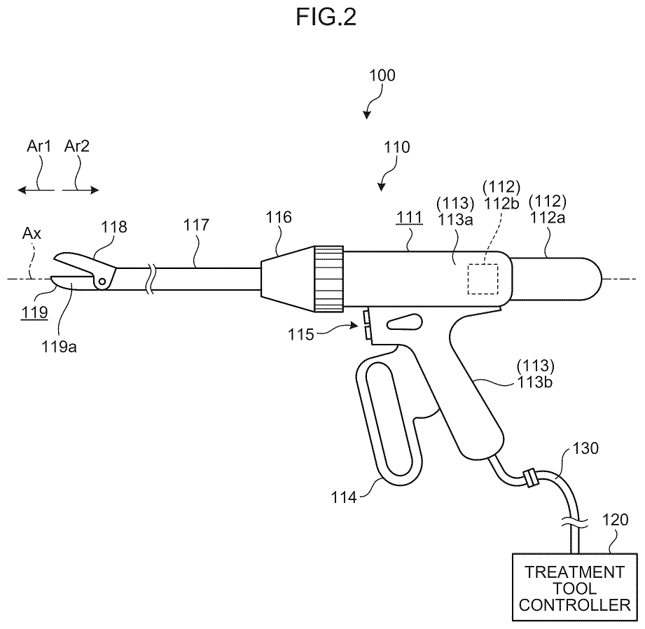

A configuration of a treatment system 100 to be connected to the endoscope system 1 described above will be described next. FIG. 2 is a diagram illustrating a schematic configuration of a treatment system to be connected to the endoscope system according to the first embodiment. One direction along a central axis Ax of a treatment tool will be referred to as a distal direction Ar1 and a direction opposite to the distal direction Ar1 as a proximal direction Ar2, as illustrated in FIG. 2.

The treatment system 100 is for treatment of a region to be treated in biological tissue (hereinafter, referred to as a target region) by application of ultrasound energy and high frequency energy to the target region. Treatment that is able to be executed by a treatment system according to this embodiment is, for example, treatment to coagulate and seal a target region, treatment to incise a target region, or treatment to perform coagulation and incision at the same time. The treatment system 100 includes a treatment tool 110 and a treatment tool controller 120.

The treatment tool 110 is an ultrasonic treatment tool for treatment of a target region by application of ultrasound energy and high frequency energy to the target region and corresponds to a surgical operation device. The treatment tool 110 includes a handpiece 111 and an ultrasound transducer unit 112.

The handpiece 111 includes a holding case 113, a movable handle 114, a switch 115, a rotation knob 116, a pipe 117, a jaw 118, and a vibration transmission member 119.

The ultrasound transducer unit 112 includes a transducer (TD) case 112a and an ultrasound transducer 112b.

The TD case 112a supports the ultrasound transducer 112b and is detachably connected to a holding case main body 113a.

The ultrasound transducer 112b generates ultrasonic vibration under control by the treatment tool controller 120. In this embodiment, the ultrasound transducer 112b is a bolted Langevin transducer (BLT).

The holding case 113 forms the external appearance of the treatment tool 110 and supports the whole treatment tool 110. The holding case 113 includes the holding case main body 113a having an approximately cylindrical shape coaxial with the central axis Ax, and a fixed handle 113b that extends downward in FIG. 2 from the main body of the holding case 113 and that is held by an operator, such as an operating surgeon.

The movable handle 114 receives opening and closing operation by the operator, such as an operating surgeon. The opening and closing operation is operation to open and close the jaw 118 relatively to an end portion 119a of the vibration transmission member 119, the end portion 119a being in the distal direction Ar1.

The switch 115 is provided in a state of being exposed externally from a side surface of the fixed handle 113b, the side surface being in the distal direction Ar1. The switch 115 receives treatment operation by the operator, such as an operating surgeon. The treatment operation is operation for applying ultrasound energy and high frequency energy to a target region. In a case where the switch 115 has plural buttons, operation instructions are assigned respectively to these buttons.

The rotation knob 116 has an approximately cylindrical shape coaxial with the central axis Ax and is provided near an end of the holding case main body 113a, the end being in the distal direction Ar1. The rotation knob 116 receives rotation operation by the operator, such as an operating surgeon. The rotation operation rotates the rotation knob 116 about the central axis Ax relatively to the holding case main body 113a. Furthermore, the rotation of the rotation knob 116 rotates the pipe 117, the jaw 118, and the vibration transmission member 119 about the central axis Ax.

The pipe 117 is a cylindrical pipe. A pin (not illustrated in the drawings) that supports the jaw 118 rotatably about the pin is fixed to an end portion of the pipe 117, the end portion being in the distal direction Ar1.

At least part of the jaw 118 includes an electrically conductive material. According to holding operation on the movable handle 114 by the operator, such as an operating surgeon, the jaw 118 is opened or closed relatively to the end portion 119a of the vibration transmission member 119, the end portion 119a being in the distal direction Ar1, and the jaw 118 holds a target region between the jaw 118 and the end portion 119a.

The vibration transmission member 119 includes an electrically conductive material and has an elongated shape extending in a straight line along the central axis Ax. Furthermore, the vibration transmission member 119 is inserted in and through the pipe 117, with the end portion 119a protruding externally, the end portion 119a being in the distal direction Ar1. An end portion of the vibration transmission member 119 is mechanically connected to the ultrasound transducer unit 112, although specific illustration of this mechanical connection has been omitted, the end portion being in the proximal direction Ar2. That is, the vibration transmission member 119 transmits ultrasonic vibration generated by the ultrasound transducer unit 112 to the end portion 119a in the distal direction Ar1 from the end portion of the vibration transmission member 119, the end portion being in the proximal direction Ar2. In this embodiment, the ultrasonic vibration is longitudinal vibration along the central axis Ax.

The treatment tool controller 120 integrally controls operation of the treatment tool 110 via an electric cable 130.

Specifically, the treatment tool controller 120 detects treatment operation on the switch 115 by the operator, such as an operating surgeon, via the electric cable 130. In a case where the treatment operation has been detected, the treatment tool controller 120 applies, via the electric cable 130, ultrasound energy or high frequency energy to a target region held between the jaw 118 and the end portion 119a of the vibration transmission member 119, the end portion 119a being in the distal direction Ar1. That is, the treatment tool controller 120 executes treatment of the target region.

For example, in applying ultrasound energy to a target region, the treatment tool controller 120 supplies driving electric power to the ultrasound transducer 112b via the electric cable 130. The ultrasound transducer 112b thereby generates longitudinal vibration (ultrasonic vibration) along the central axis Ax. The end portion 119a of the vibration transmission member 119 vibrates at a desired amplitude due to the longitudinal vibration, the end portion 119a being in the distal direction Ar1. The ultrasonic vibration is thus applied from the end portion 119a to the target region held between the jaw 118 and the end portion 119a. In other words, the ultrasound energy is applied to the target region from the end portion 119a. Furthermore, for example, in applying high frequency energy to a target region, the treatment tool controller 120 supplies high frequency electric power between the jaw 118 and the vibration transmission member 119 via the electric cable 130. High frequency electric current thereby flows to the target region held between the jaw 118 and the end portion 119a of the vibration transmission member 119, the end portion 119a being in the distal direction Ar1. In other words, the high frequency energy is applied to the target region.

The treatment tool controller 120 is connected to the control device 9 to be able to communicate with the control device 9 and outputs, in response to the switch 115 being pressed down, a signal indicating that a switch has been pressed down.

### Functional Configuration of Main Parts of Endoscope System

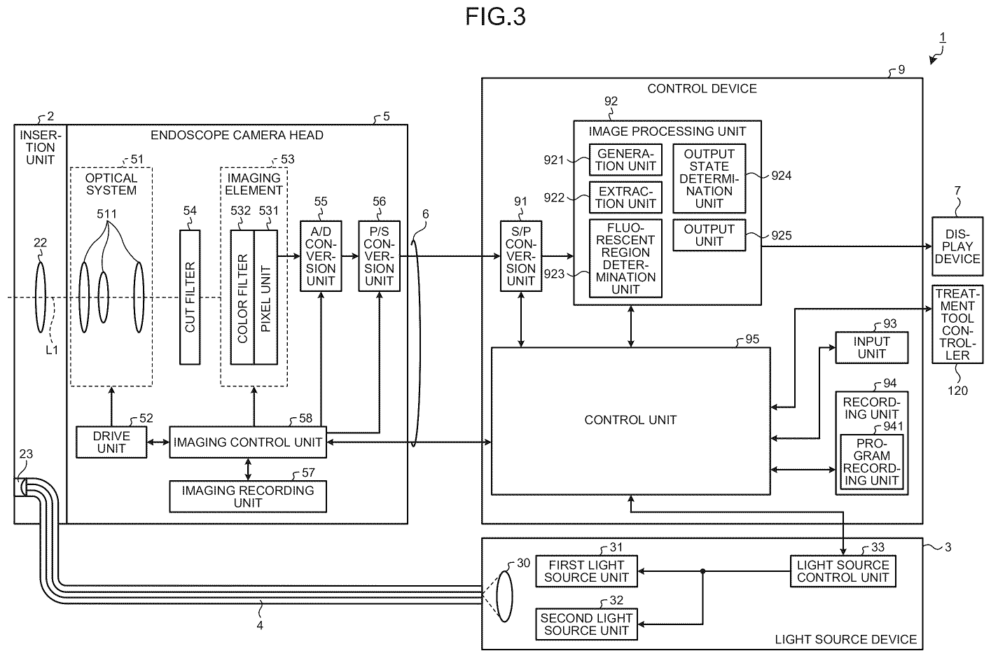

A functional configuration of main parts of the endoscope system 1 described above will be described next. FIG. 3 is a block diagram illustrating the functional configuration of the main parts of the endoscope system 1.

### Configuration of Insertion Unit

A configuration of the insertion unit 2 will be described first. The insertion unit 2 has an optical system 22 and an illumination optical system 23.

The optical system 22 forms a subject image by condensing light, such as reflected light reflected from a subject, returned light from the subject, excitation light from the subject, and emitted light emitted by the subject. The optical system 22 is implemented using one or plural lenses, for example.

Illumination light supplied from the light guide 4 is emitted from the illumination optical system 23 to a subject. The illumination optical system 23 is implemented using one or plural lenses, for example.

### Configuration of Light Source Device

A configuration of the light source device 3 will be described next. The light source device 3 includes a condenser lens 30, a first light source unit 31, a second light source unit 32, and a light source control unit 33.

Light emitted by each of the first light source unit 31 and the second light source unit 32 is condensed and output to the light guide 4 by the condenser lens 30.

Under control by the light source control unit 33, the first light source unit 31 supplies, as illumination light, white light (normal light), which is visible light, to the light guide 4 by emitting the white light. The first light source unit 31 is formed using a collimator lens, a white LED lamp, and a driver, for example. The first light source unit 31 may supply white light of visible light by simultaneous light emission using a red LED lamp, a green LED lamp, and a blue LED lamp. Of course, the first light source unit 31 may be formed using a halogen lamp or a xenon lamp, for example.

Under control by the light source control unit 33, the second light source unit 32 supplies, as illumination light, narrowband light in a wavelength band different from and narrower than that of the white light, to the light guide 4 by emitting the narrowband light. The narrowband light is, for example, light in a wavelength band of 400 nm to 430 nm having a center wavelength of 415 nm. The second light source unit 32 is implemented using a collimator lens, a semiconductor laser, such as a violet laser diode (LD), and a driver, for example. In this embodiment, the narrowband light functions as excitation light that excites advanced glycation end-products produced by heat treatment on biological tissue.

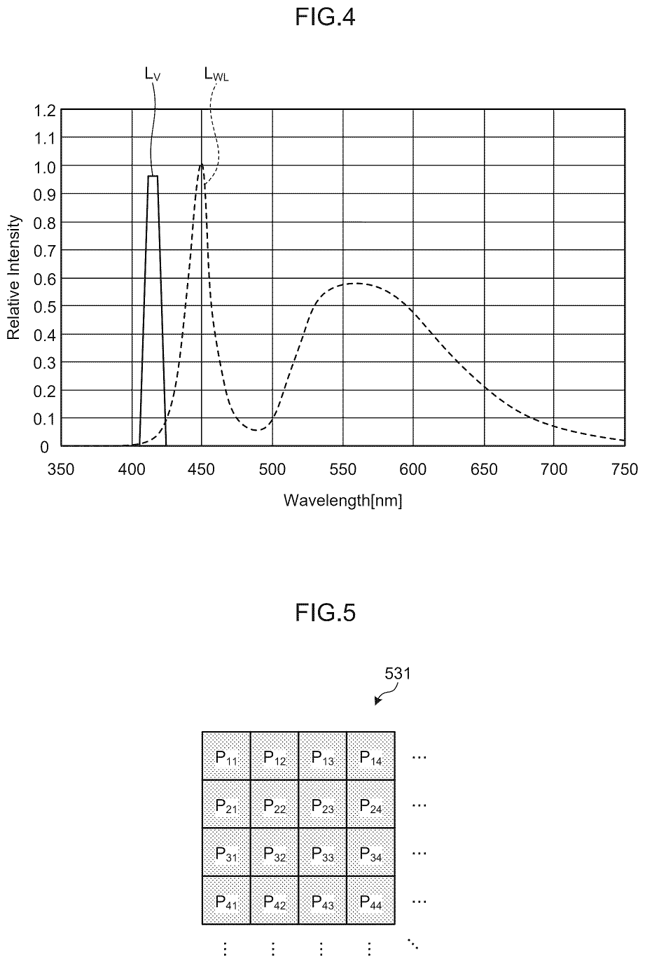

The light source control unit 33 is implemented using: a processor that is a processing device having hardware, such as an FPGA or a CPU; and a memory that is a temporary storage area used by the processor. On the basis of control data input from the control device 9, the light source control unit 33 controls light emission timing and light emission time periods, for example, of the first light source unit 31 and the second light source unit 32. The following description is on wavelength characteristics of light emitted by the first light source unit 31 and the second light source unit 32. FIG. 4 is a diagram schematically illustrating the wavelength characteristics of the light emitted by each of the first light source unit 31 and the second light source unit 32. In FIG. 4, the horizontal axis represents wavelength (nm) and the vertical axis represents relative intensity. In FIG. 4, a curve L_{WL} represents the wavelength characteristics of the white light emitted by the first light source unit 31 and a curve L_{V} represents the wavelength characteristics of the narrowband light (excitation light) emitted by the second light source unit 32. The second light source unit 32 emits light including the wavelength band of 400 nm to 430 nm, with the center wavelength (peak wavelength) of 415 nm. The wavelength characteristics represented by the curve L_{WL} in FIG. 4 represent characteristics in a case where a white LED is adopted as the first light source unit 31.

### Configuration of Endoscope Camera Head

The configuration of the endoscope system 1 will be described further by reference to FIG. 3.

A configuration of the endoscope camera head 5 will be described next. The endoscope camera head 5 includes an optical system 51, a drive unit 52, an imaging element 53, a cut filter 54, an A/D conversion unit 55, a P/S conversion unit 56, an imaging recording unit 57, and an imaging control unit 58.

The optical system 51 forms a subject image condensed by the optical system 22 of the insertion unit 2 on a light receiving surface of the imaging element 53. The focal length and focal position of the optical system 51 are changeable. The optical system 51 is formed using plural lenses 511. The focal length and focal position of the optical system 51 are changed by the drive unit 52 moving each of the plural lenses 511 on an optical axis L1.

Under control by the imaging control unit 58, the drive unit 52 moves the plural lenses 511 of the optical system 51 along and on the optical axis L1. The drive unit 52 is formed using: a motor, such as a stepping motor, a DC motor, or a voice coil motor; and a transmission mechanism, such as a gear, which transmits rotation of the motor to the optical system 51.

The imaging element 53 is implemented using an image sensor, such as a charge coupled device (CCD) or a complementary metal oxide semiconductor (CMOS), which has plural pixels arranged in a two-dimensional matrix. Under control by the imaging control unit 58, the imaging element 53 optically receives a subject image via the cut filter 54, generates an imaging signal (RAW data) by photoelectric conversion of the subject image, and outputs the imaging signal to the A/D conversion unit 55, the subject image being a subject image (light rays) formed by the optical system 51. The imaging element 53 has a pixel unit 531 and a color filter 532.

FIG. 5 is a diagram schematically illustrating a configuration of the pixel unit 531. The pixel unit 531 has plural pixels Pₙₘ (where n and m are whole numbers equal to or larger than 1), such as photodiodes that accumulate electric charges corresponding to quantities of light, arranged in a two-dimensional matrix. Under control by the imaging control unit 58, the pixel unit 531 reads and outputs, as image data, image signals from pixels Pₙₘ in a read area optionally set as a target to be read, to the A/D conversion unit 55, the pixels Pₙₘ being from the plural pixels Pₙₘ.

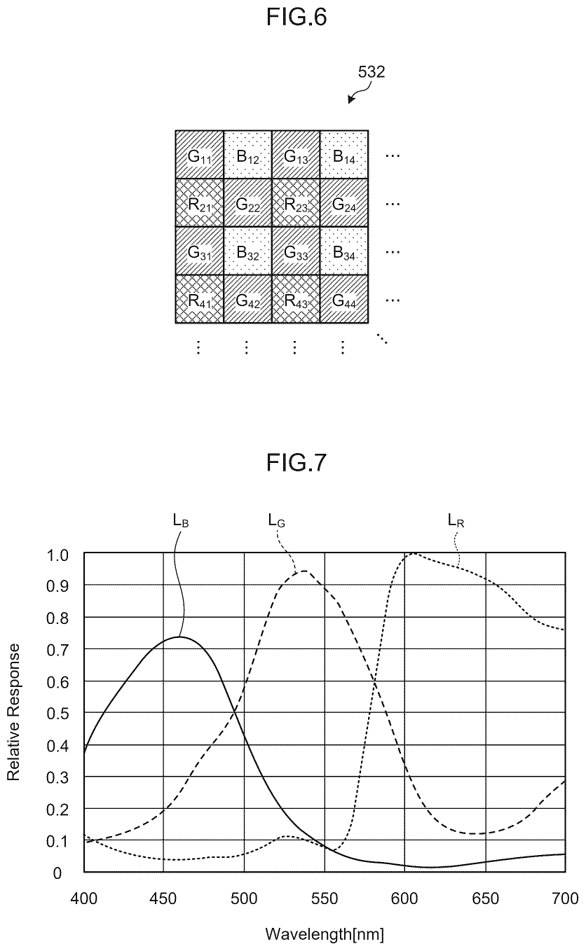

FIG. 6 is a diagram schematically illustrating a configuration of the color filter 532. The color filter 532 has a Bayer array in units of 2×2. The color filter 532 is formed using a filter R that transmits light in a red wavelength band, two filters G that transmit light in a green wavelength band, and a filter B that transmits light in a blue wavelength band. In FIG. 6, a reference numeral (for example, G₁₁) assigned to each filter corresponds to a pixel Pₙₘ and indicates that the filter is arranged at a position of its corresponding pixel.

FIG. 7 is a diagram schematically illustrating sensitivity characteristics of each filter. In FIG. 7, the horizontal axis represents wavelength (nm) and the vertical axis represents transmission characteristics (sensitivity characteristics). Furthermore, in FIG. 7, a curve L_{B} represents transmission characteristics of the filter B, a curve L_{G} represents transmission characteristics of the filter G, and a curve L_{R} represents transmission characteristics of the filter R.

The filter B transmits light in the blue wavelength band (see the curve L_{B} in FIG. 7). The filter G transmits light in the green wavelength band (see the curve L_{G} in FIG. 7). The filter R transmits light in the red wavelength band (see the curve L_{R} in FIG. 7). A pixel Pₙₘ having a filter R arranged on a light receiving surface thereof will hereinafter be referred to as an R pixel, a pixel Pₙₘ having a filter G arranged on a light receiving surface thereof as a G pixel, and a pixel Pₙₘ having a filter B arranged on a light receiving surface thereof as a B pixel.

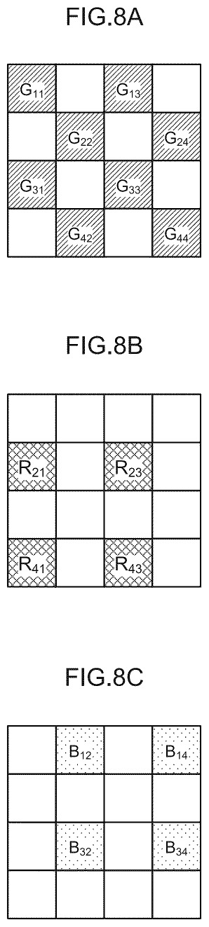

In a case where a subject image formed by the optical system 51 is optically received by the imaging element 53 configured as described above, color signals (R signals, G signals, and B signals) of R pixels, G pixels, and B pixels are generated (see FIG. 8A to FIG. 8C).

The configuration of the endoscope system 1 will be described further by reference to FIG. 3.

The cut filter 54 is arranged on the optical axis L1 of the optical system 51 and the imaging element 53. The cut filter 54 is provided on a light receiving surface side (incident surface side) of at least the G pixels having, provided thereon, the filters G of the color filter 532, the filters G being filters that transmit the green wavelength band therethrough. The cut filter 54 blocks light in the wavelength band of the excitation light and transmits light in a wavelength band longer in wavelength than the wavelength band of the excitation light.

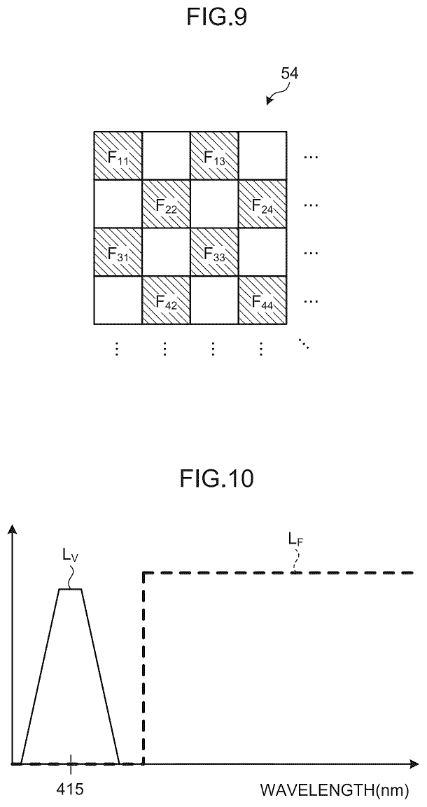

FIG. 9 is a diagram schematically illustrating a configuration of the cut filter 54. As illustrated in FIG. 9, a filter F₁₁ included in the cut filter 54 is arranged at a position where the filter G₁₁ (see FIG. 6) is arranged, the position being on a light receiving surface side of the filter G₁₁ and being immediately above the filter G₁₁.

FIG. 10 is a diagram schematically illustrating transmission characteristics of the cut filter 54. In FIG. 10, the horizontal axis represents wavelength (nm) and the vertical axis represents the transmission characteristics. Furthermore, in FIG. 10, a curve L_{F} represents the transmission characteristics of the cut filter 54 and a curve L_{V} represents wavelength characteristics of the excitation light.

The cut filter 54 blocks the wavelength band of the excitation light and transmits therethrough the wavelength band longer in wavelength than the wavelength band of the excitation light. Specifically, the cut filter 54 blocks light in the wavelength band of the excitation light and light shorter in wavelength than the wavelength band of the excitation light and transmits therethrough light in a wavelength band longer in wavelength than the excitation light.

The configuration of the endoscope camera head 5 will be described further by reference to FIG. 3.

Under control by the imaging control unit 58, the A/D conversion unit 55 performs A/D conversion processing of an analog imaging signal input from the imaging element 53 and outputs a digital imaging signal resulting from the A/D conversion processing to the P/S conversion unit 56. The A/D conversion unit 55 is implemented using an A/D conversion circuit, for example.

Under control by the imaging control unit 58, the P/S conversion unit 56 performs parallel/serial conversion of the digital imaging signal input from the A/D conversion unit 55 and outputs an imaging signal resulting from the parallel/serial conversion to the control device 9 via the first transmission cable 6. The P/S conversion unit 56 is implemented using a P/S conversion circuit, for example. In the first embodiment, an E/O conversion unit that converts an imaging signal to an optical signal may be provided instead of the P/S conversion unit 56 and the imaging signal may be output to the control device 9 by means of the optical signal, or, an imaging signal may be transmitted to the control device 9 by wireless communication of Wireless Fidelity (Wi-Fi) (registered trademark), for example.

The imaging recording unit 57 records therein various kinds of information related to the endoscope camera head 5 (for example, pixel information on the imaging element 53 and characteristics of the cut filter 54). Furthermore, the imaging recording unit 57 records therein various set data and control parameters transmitted from the control device 9 via the first transmission cable 6. The imaging recording unit 57 is formed using a non-volatile memory and/or a volatile memory.

On the basis of set data received from the control device 9 via the first transmission cable 6, the imaging control unit 58 controls operation of each of the drive unit 52, the imaging element 53, the A/D conversion unit 55, and the P/S conversion unit 56. The imaging control unit 58 is implemented using: a timing generator (TG); a processor that is a processing device having hardware, such as a CPU; and a memory that is a temporary storage area used by the processor.

### Configuration of Control Device

A configuration of the control device 9 will be described next.

The control device 9 includes an S/P conversion unit 91, an image processing unit 92, an input unit 93, a recording unit 94, and a control unit 95.

Under control by the control unit 95, the S/P conversion unit 91 performs serial/parallel conversion of image data received from the endoscope camera head 5 via the first transmission cable 6 and outputs the converted image data to the image processing unit 92. In a case where the endoscope camera head 5 outputs an imaging signal by means of an optical signal, an O/E conversion unit that converts an optical signal to an electric signal may be provided instead of the S/P conversion unit 91. In a case where the endoscope camera head 5 transmits an imaging signal by wireless communication, a communication module capable of receiving a wireless signal may be provided instead of the S/P conversion unit 91.

Under control by the control unit 95, the image processing unit 92 performs predetermined image processing of the imaging signal that is parallel data input from the S/P conversion unit 91 and outputs the processed imaging signal to the display device 7. The predetermined image processing is, for example, demosaicing, white balance processing, gain adjustment, γ correction, and format conversion. The image processing unit 92 is implemented using: a processor that is a processing device having hardware, such as a GPU or an FPGA; and a memory that is a temporary storage area used by the processor. The image processing unit 92 has a generation unit 921, an extraction unit 922, a fluorescent region determination unit 923, an output state determination unit 924, and an output unit 925.

The generation unit 921 generates a first image including one or more feature regions required to be excised by an operating surgeon, and a second image including one or more cauterized regions cauterized by an energy device (the treatment tool 110). Specifically, the generation unit 921 generates a white light image that is the first image, on the basis of an imaging signal generated by imaging of reflected light and returned light from biological tissue upon irradiation of the biological tissue with white light. Furthermore, the generation unit 921 generates a fluorescence image that is the second image, on the basis of an imaging signal generated by imaging of fluorescence generated by excitation light emitted for excitation of advanced glycation end-products produced by heat treatment on the biological tissue in a fluorescence observation mode described later. The generation unit 921 may generate a pseudo-color image including one or more feature regions (lesion regions) required to be excised by an operating surgeon, on the basis of an imaging signal resulting from imaging of reflected light and returned light from the biological tissue upon irradiation of the biological tissue with excitation light in the fluorescence observation mode of the endoscope system 1 described later.

The extraction unit 922 extracts a fluorescent region that is a region of a fluorescent image, from the fluorescence image generated by the generation unit 921.

The fluorescent region determination unit 923 determines whether or not there is a change in fluorescent regions between fluorescence images imaged at different times.

The output state determination unit 924 determines an output state of the treatment tool 110 on the basis of a signal that the control device 9 received from the treatment tool controller 120. Specifically, the output state determination unit 924 determines whether output of the treatment tool 110 is in an on-state or in an off-state.

On the basis of results of the determination by the fluorescent region determination unit 923 and the output state determination unit 924, the control unit 95 sets the relevant fluorescent region as a thermal denaturation region (off-time generated fluorescent region) that has been generated during the off-state of output of the energy device (treatment tool 110).

The output unit 925 outputs, for example, a white light image, a fluorescence image, a result of determination by the fluorescent region determination unit 923, and set information set by the control unit 95.

The input unit 93 receives input of various kinds of operation related to the endoscope system 1 and outputs the operation received to the control unit 95. The input unit 93 is formed using any of a mouse, a foot switch, a keyboard, a button, a switch, and a touch panel, for example.

The recording unit 94 is implemented using any of recording media, such as a volatile memory, a non-volatile memory, a solid state drive (SSD), a hard disk drive (HDD), and a memory card. The recording unit 94 records therein data including various parameters required in operation of the endoscope system 1. The recording unit 94 has a program recording unit 941 that records therein various programs for operating the endoscope system 1.

The control unit 95 is implemented using: a processor that is a processing device having hardware, such as an FPGA or a CPU; and a memory that is a temporary storage area used by the processor. The control unit 95 integrally controls the components of the endoscope system 1. Furthermore, the control unit 95 receives a signal related to the switch 115 being pressed down (output from the treatment tool 110), from the treatment tool controller 120.

### Outline of Each Observation Mode

An outline of each observation mode that is able to be executed in the endoscope system 1 will be described next. A normal light observation mode and the fluorescence observation mode will be described hereinafter in this order.

### Outline of Normal Light Observation Mode

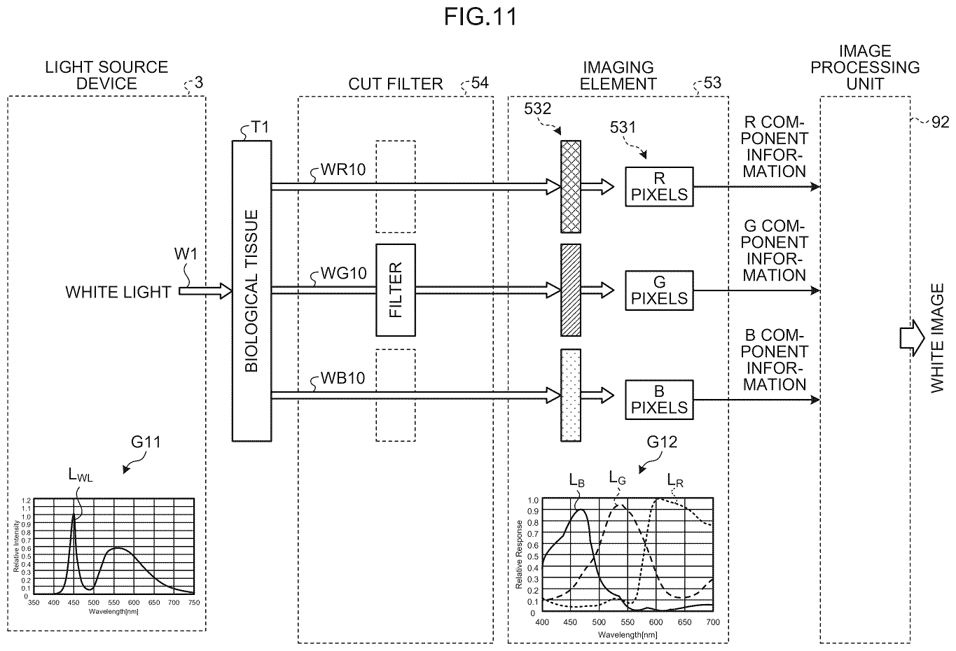

The normal light observation mode will be described first. FIG. 11 is a diagram schematically illustrating observation principles for the normal light observation mode.

Under control by the control device 9, the light source device 3 emits white light W1 having an intensity distribution represented by a graph G11 to biological tissue T1 of a subject by causing light emission by the first light source unit 31. In this case, part of reflected light and returned light reflected by the biological tissue T1 (hereinafter, simply referred to as “reflected light WR10, reflected light WG10, and reflected light WB10”) is blocked by the cut filter 54 and the rest of the reflected light and returned light enters the imaging element 53. For example, specifically, the cut filter 54 blocks reflected light (reflected light WG10) that is incident on the G pixels and that is in the wavelength band of excitation light (excitation light W2 described later). That is, reflected light and returned light that are based on the irradiation with the white light W1 are incident on the filters R and the filters B, and light in a wavelength band longer in wavelength than the wavelength band of the excitation light is incident on the filters G. Therefore, the light component in the blue wavelength band incident on the pixels is less than that in a state where the cut filter 54 has not been arranged. Light incident on each filter is selectively transmitted according to filter characteristics represented by a graph G12.

Subsequently, the image processing unit 92 acquires image data (RAW data) from the imaging element 53 of the endoscope camera head 5, performs image processing of signal values of R pixels, G pixels, and B pixels included in the image data acquired, and thereby generates a white light image. In this case, because the blue component included in the image data is less than that in conventional white light observation, the image processing unit 92 performs white balance adjustment processing for adjustment of white balance so that the ratio between the red component, green component, and blue component becomes constant.

In the normal light observation mode, a natural white light image (observation image) is able to be observed even in a case where the cut filter 54 has been arranged on the light receiving surface side of the G pixels.

### Outline of Fluorescence Observation Mode

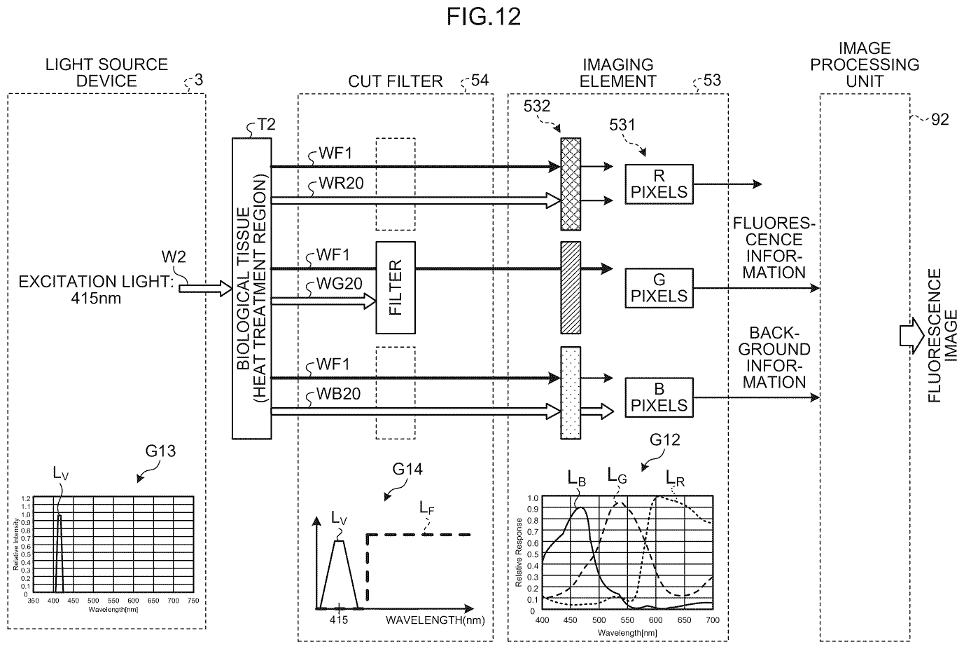

The fluorescence observation mode will be described next. FIG. 12 is a diagram schematically illustrating observation principles for the fluorescence observation mode.

Minimally invasive treatment using endoscope and laparoscopes, for example, has been increasingly implemented in the medical field recently. For example, endoscopic submucosal dissection (ESD), laparoscopy and endoscopy cooperative surgery (LECS), non-exposed endoscopic wall-inversion surgery (NEWS), and transurethral resection of the bladder tumor (TUR-bt) have been widely implemented as minimally invasive treatment using devices, such as endoscope and laparoscopes.

In performing treatment in such minimally invasive treatment, for example, an operating surgeon, such as a medical doctor, performs heat treatment using a treatment tool that is an energy device that emits energy of a high frequency, ultrasound, or microwaves, performs marking of a surgical target region as pretreatment, and performs, as the treatment, excision of a lesion or sealing or coagulation of an incision.

Heating an amino compound and a reducing sugar causes a glycation reaction (Maillard reaction), in which amino acids and the reducing sugar react. End products produced by this Maillard reaction are generally called advanced glycation end products (AGEs). One known characteristic of these AGEs is that they include fluorescent substances. AGEs are known to emit fluorescence of an intensity higher than that of autofluorescent substances naturally found in biological tissue. Therefore, the production of AGEs increases the intensity of fluorescence as compared to that before the production of AGES.

AGEs produced by cauterization in treatment are able to be visualized through observation of fluorescence, and intensity of the fluorescence serves as an index of a state of heat treatment.

The is, the fluorescence observation mode is an observation mode, in which a heat treatment region is visualized by utilization of the fluorescent characteristic of AGEs produced in biological tissue by heat treatment using an energy device, for example. Therefore, in the fluorescence observation mode, the excitation light for exciting AGEs, for example, the blue narrowband light having the center wavelength of 415 nm, is emitted to biological tissue from the light source device 3. The fluorescence observation mode thereby enables observation of a heat treatment image (fluorescence image) resulting from imaging of fluorescence (for example, green light having wavelengths of 490 to 625 nm) generated by AGEs.

Specifically, under control by the control device 9, firstly, the light source device 3 emits excitation light W2 (having a center wavelength of 415 nm: see a graph G13) to biological tissue T2 (heat treatment region) of a subject subjected to heat treatment using an energy device, for example, by causing light emission by the second light source unit 32. In this case, at least reflected light including components of the excitation light W2 and returned light reflected by the biological tissue T2 (heat treatment region) (hereinafter, simply referred to as “reflected light WR20, reflected light WG20, and reflected light WB20”) is blocked by the cut filter 54 and part of longer wavelength components is incident on the imaging element 53 (see a graph G14). In FIG. 12, the intensity (quantity of light or signal value) of each component is represented by the thickness of the arrow.

More specifically, as illustrated by the graph G14 in FIG. 12, the cut filter 54 blocks the reflected light WG20 to be incident on the G pixels and that is in a wavelength band including the wavelength band of the excitation light W2. Furthermore, the cut filter 54 transmits therethrough fluorescence WF1 that is autofluorescence from AGEs in the biological tissue T2 (heat treatment region) (see the graph G14). Therefore, the reflected light WG20 does not enter the G pixels and the fluorescence WF1 enters the G pixels. Because the cut filter 54 is arranged on the light receiving surface side (incident surface side) of the G pixels, the fluorescent component is able to be prevented from being buried in a mixture of the fluorescence WF1 and the reflected light WG20 of the excitation light W2.

The reflected light WR20 and fluorescence WF1 enter the R pixels and the reflected light WB20 and fluorescence WF1 enter the B pixels.

Thereafter, the image processing unit 92 acquires image data (RAW data) from the imaging element 53 of the endoscope camera head 5, performs image processing of signal values of G pixels and B pixels included in the image data acquired, and thereby generates a fluorescence image. In this case, the signal values of the G pixels include fluorescence information representing a fluorescent image generated from the heat treatment region. The B pixels include background information on the background of the heat treatment region, the background being biological tissue around the heat treatment region. The image processing unit 92 generates the fluorescence image by performing image processing, such as gain control processing, pixel interpolation, and mucosal enhancement, of the signal values of the G pixels and B pixels included in the image data. In the gain control processing, the image processing unit 92 performs processing to make gains of the signal values of the G pixels larger than gains of signal values of G pixels in normal light observation and make gains of the signal values of the B pixels smaller than gains of signal values of B pixels in the normal light observation. Furthermore, the image processing unit 92 executes processing so that the signal values of the G pixels and the signal values of the B pixels become the same (1:1). The image processing unit 92 may generate a pseudo-color image having color information superposed on a fluorescent image, the color information having hues changed according to intensity of fluorescence.

### Treatment Using Endoscope System

Treatment using the endoscope system 1 according to the present disclosure will be described next. Upon the treatment, an operating surgeon inserts the insertion unit 2 in a subject and causes the light source device 3 to emit white light to the interior of the subject to irradiate a region including a treatment target with white light. While observing an observation image displayed by the display device 7, the operating surgeon checks the treatment target.

Thereafter, while checking a white light image displayed by the display device 7, the operating surgeon performs the treatment of the treatment target in the subject. For example, the operating surgeon subjects the treatment target to cauterization and excision using a device, such as an energy device (the treatment tool 110) inserted in the subject via the insertion unit 2.

Thereafter, the operating surgeon irradiates the treatment target with excitation light and observes a fluorescence image displayed by the display device 7. By observing the fluorescence image displayed by the display device 7, the operating surgeon determines whether or not the treatment (for example, the excision) at the treatment position has been completed. In a case where the operating surgeon determines that the treatment has been completed, the operating surgeon ends the manipulation. Specifically, the operating surgeon observes the fluorescence image displayed by the display device 7 to observe a cauterized region excised through cauterization by means of the treatment tool 110 and thereby determines whether or not the excision of the treatment target has been completed. In a case where the operating surgeon determines that the excision of the treatment target has not been completed, the operating surgeon continues the treatment by repeating the observation of the white light image through the irradiation with the white light and the observation of the fluorescence image through the irradiation with the excitation light, while switching the observation modes of the endoscope system 1.

### Process in Endoscope System

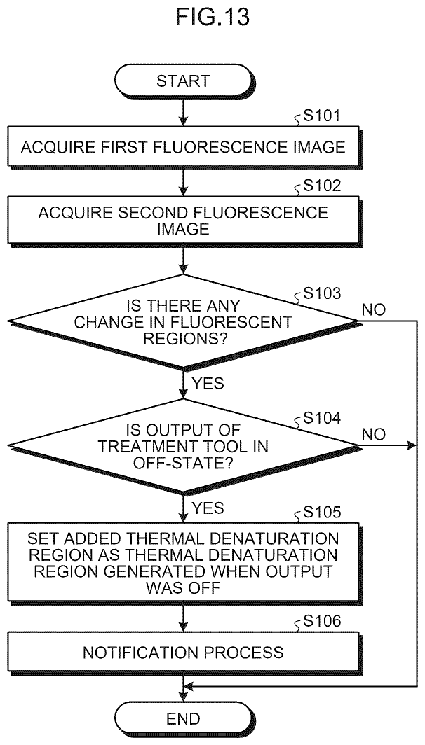

A process executed by the endoscope system 1 will be described next. FIG. 13 is a flowchart illustrating a thermal denaturation region determination process using an endoscope system according to an embodiment. The thermal denaturation region determination process is a process executed in the fluorescence observation mode.

The control unit 95 generates a first fluorescence image (Step S101). In generating the first fluorescence image, the control unit 95 controls the light source control unit 33 to cause light emission by the second light source unit 32 and to irradiate a subject with excitation light. By acquiring an imaging signal from the imaging element 53 of the endoscope camera head 5, the generation unit 921 generates the first fluorescence image. The first fluorescence image is thereby acquired. In this case, the output unit 925 may cause the display device 7 to display the first fluorescence image generated by the generation unit 921.

Subsequently, the control unit 95 generates a second fluorescence image (Step S102). In generating the second fluorescence image, the control unit 95 controls the light source control unit 33 to cause light emission by the second light source unit 32 and to irradiate a subject with excitation light. By acquiring an imaging signal from the imaging element 53 of the endoscope camera head 5, the generation unit 921 generates the second fluorescence image. The second fluorescence image is thereby acquired. In this case, the output unit 925 may cause the display device 7 to display the second fluorescence image generated by the generation unit 921.

The second fluorescence image is a fluorescence image based on image data acquired at a time after that of the first fluorescence image. The acquisition (imaging) of the image data is executed after elapse of a preset time period from the acquisition of the first fluorescence image, for example.

Thereafter, the control unit 95 determines whether or not there is a change in fluorescent regions between the first fluorescence image and the second fluorescence image (Step S103). At this step, the extraction unit 922 extracts a region (fluorescent region) representing an image of fluorescence from each of these fluorescence images. For example, the extraction unit 922 extracts one or plural fluorescent regions included in an image by executing contour extraction based on luminance values. The fluorescent region determination unit 923 then determines whether or not there is a change in the fluorescent regions in the second fluorescence image from the extracted fluorescent regions in the first fluorescence image. In this determination, the fluorescent region determination unit 923 detects a change in the fluorescent regions by determining whether or not there is any new fluorescent region that is present in the second fluorescence image but not present in the first fluorescence image. In a case where the fluorescent region determination unit 923 determines that there is no change in the fluorescent regions (Step S103: No), the control unit 95 ends the process. By contrast, in a case where the fluorescent region determination unit 923 determines that there is a change in the fluorescent regions (Step S103: Yes), the control unit 95 proceeds to Step S104.

At Step S104, the control unit 95 determines whether or not the output state of the treatment tool 110 is the off-state. At this step, the output state determination unit 924 determines whether or not the treatment tool 110 at the time of imaging of the second fluorescence image is in the off-state or the on-state. For example, on the basis of a signal from the treatment tool controller 120 and received by the control device 9, the output state determination unit 924 determines whether the output by the treatment tool 110 is on or off at the imaging time of the second fluorescence image. If each of the fluorescence images has been provided with information on either the on-state or the off-state of the output by the treatment tool 110, the output state determination unit 924 determines either the on-state or the off-state by referring to that information. In a case where the output state determination unit 924 determines that the output state of the treatment tool 110 is the on-state, that is, not the off-state (Step S104: No), the control unit 95 ends the process. By contrast, in a case where the output state determination unit 924 determines that the output state of the treatment tool 110 is the off-state (Step S104: Yes), the control unit 95 proceeds to Step S105.

At Step S105, the control unit 95 sets the thermal denaturation region (new fluorescent region) added in the second fluorescence image as a thermal denaturation region (off-time generated fluorescent region) that has been generated during the off-state of output of the treatment tool 110. In this setting, the control unit 95 sets, as the thermal denaturation region that has been generated during the off-state of output of the treatment tool 110, a fluorescent region not present in the first fluorescence image, the fluorescent region being one of fluorescent regions extracted by the extraction unit 922 from the second fluorescence image.

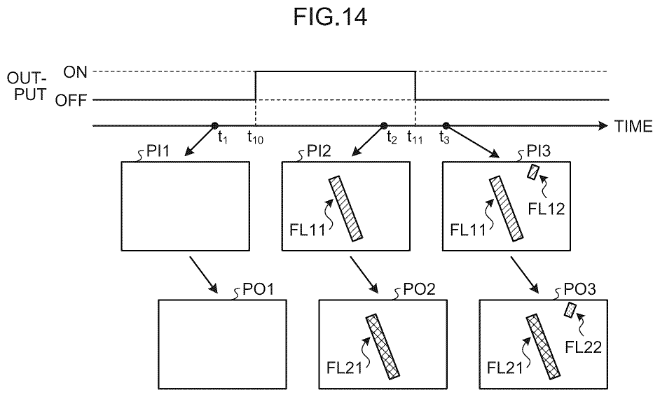

Temporal changes in fluorescence images will now be described by reference to FIG. 14. FIG. 14 is a diagram for description of fluorescent images in the fluorescence observation mode. FIG. 14 illustrates an example, in which the output of the treatment tool 110 is turned on at a time t₁₀ and the output of the treatment tool 110 is turned off at a time t₁₁.

In FIG. 14, for example, at a time t₁ before the time t₁₀, even if a treatment region is irradiated with excitation light, no AGEs are present and no fluorescent image is depicted (see an image PI1). Therefore, even if a fluorescence image at that time is displayed on the display device 7, no fluorescent image will be displayed (see an image PO1).

Thereafter, for example, at a time t₂ after the time t₁₀ and before the time t₁₁, irradiating the treatment region with excitation light causes a fluorescent image FL11 to be depicted, the fluorescent image FL11 corresponding to AGEs (see an image PI2). Therefore, when a fluorescence image at that time is displayed on the display device 7, a fluorescent image FL21 is displayed (see an image PO2). This fluorescent image FL11 (FL21) corresponds to, for example, AGEs generated by energy applied from the treatment tool 110.

The fluorescent image FL11 and the fluorescent image FL21 may be displayed with the same hues, or, for example, an image having pseudo-colors superposed on the fluorescent image FL21 may be displayed.

Thereafter, for example, new AGEs are produced by the treatment tool 110 contacting biological tissue at a time t₃ after the time t₁₁, output of the treatment tool 110 being in the off-state, and a fluorescent image FL12 corresponding to these new AGEs is depicted (see an image PI3). Therefore, when a fluorescence image at that time is displayed on the display device 7, a fluorescent image FL22 is displayed (see an image PO3). This fluorescent image FL12 (FL22) corresponds to, for example, AGEs produced by remaining heat from the treatment tool 110.

This newly generated fluorescent image FL12 is detected as a change in the fluorescent regions by the fluorescent region determination unit 923 and is set as a thermal denaturation region that has been generated during the off-state of output of the treatment tool 110. The fluorescent image FL22 displayed on the display device 7 can be provided with information indicating that the fluorescent image FL22 has been generated during the off-state of output of the treatment tool 110 or can have a hue superposed thereon and indicating that the fluorescent image FL22 has been generated during the off-state of output of the treatment tool 110. Only the thermal denaturation region (the fluorescent image FL22 in FIG. 14) generated after the output was turned off may be displayed in a display mode to enable the thermal denaturation region to be known.

As illustrated in FIG. 13, after the thermal denaturation region has been set, the control unit 95 executes a notification process for the thermal denaturation region generated when the output was off (Step S106). In this notification process, the control unit 95 displays information indicating that the thermal denaturation region has been generated during the off-state of output of the treatment tool 110, on the display device 7. For example, textual information indicating that a thermal denaturation region was newly generated when the output was off is displayed with a fluorescence image to be compared with displayed together, or textual information or a pseudo-color assigned according to whether the output was on or off is displayed superposed on the relevant fluorescent region on the second fluorescence image, the textual information indicating that the relevant fluorescent region is a thermal denaturation region generated when the output was off. According to any condition set for the notification process, the generation unit 921 generates an image for display as described above. Information corresponding to the fluorescent region may be displayed on the white light image, or a notification of the generation of the thermal denaturation region during the off-state of output of the treatment tool 110 may be made by means of sound and/or light.

The thermal denaturation region determination process is executed at, for example, preset time intervals, or a time when an operating surgeon inputs an instruction to execute a detection process, for example. In this process, the second fluorescence image acquired in the last process may serve as a first fluorescence image and in that case, the process may be started from Step S102.

In the first embodiment described above, in a case where a change has been generated in fluorescent regions in a fluorescence image, an output state of a treatment tool at a time of imaging of the fluorescence image with the generated change is determined, this newly generated fluorescent region is set as a fluorescent region (off-time generated fluorescent region) corresponding to a thermal denaturation region that has been generated during the off-state of output of the energy device, and a notification is made to an operating surgeon. The first embodiment enables the operating surgeon to know about any thermal denaturation that has been generated during the off-state of output of the treatment tool.

### Modified Example

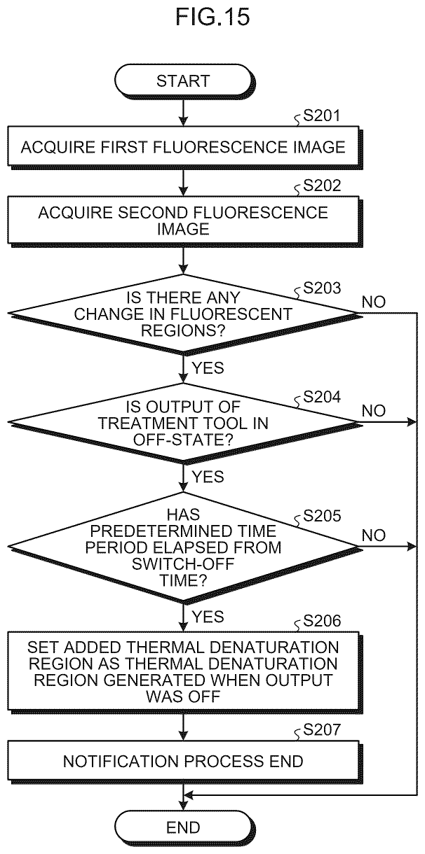

A modified example of the first embodiment will be described next by reference to FIG. 15. An endoscope system according to the modified example is similar to the endoscope system 1 according to the first embodiment and description thereof will thus be omitted. A thermal denaturation region may be enlarged due to remaining heat even after the treatment tool 110 has been separated. This region enlarged by the remaining heat is usually a thermal denaturation region already known by an operating surgeon and is thus not required to be set as a new thermal denaturation region. An example, in which this region enlarged by the remaining heat is excluded from any new thermal denaturation region generated after output has been turned off, will be described with respect to this modified example.

### Process in Endoscope System

A process executed by the endoscope system according to the modified example will be described next. FIG. 15 is a flowchart illustrating a thermal denaturation region determination process using the endoscope system according to the modified example.

Similarly to the first embodiment, the control unit 95 executes generation of first and second fluorescence images and detection of any change in fluorescent regions (Steps S201 to S203).

In a case where there is any change in the fluorescent regions (Step S203: Yes), the control unit 95 determines whether or not the output state of the treatment tool 110 is the off-state (Step S204), similarly to Step S104. In a case where the control unit 95 determines that the output of the treatment tool 110 is in the off-state at the imaging time of the second fluorescence image (Step S204: Yes), the control unit 95 proceeds to Step S205.

At Step S205, the control unit 95 determines whether or not a predetermined time period has elapsed since the time, at which the treatment tool 110 was switched off. Specifically, the control unit 95 determines whether or not the imaging time of the second fluorescence image is a time after elapse of a preset time period from the time, at which the treatment tool 110 was turned off by the switch 115 being pressed down. In a case where the control unit 95 determines that the imaging time of the second fluorescence image is not a time after elapse of a predetermined time period from the switch-off time (Step S205: No), the control unit 95 ends the process. In contrast, in a case where the control unit 95 determines that the imaging time of the second fluorescence image is a time after elapse of the predetermined time period from the switch-off time (Step S205: Yes), the control unit 95 proceeds to Step S206.

At Step S206, the control unit 95 sets the thermal denaturation region added in the second fluorescence image as a thermal denaturation region that has been generated during the off-state of output of the treatment tool 110.

After the thermal denaturation region has been set, the control unit 95 executes a notification process for the thermal denaturation region that has been generated during the off-state of output of the treatment tool 110 (Step S207). In the notification process, similarly to Step S106, the control unit 95 displays information on the display device 7, the information indicating that the thermal denaturation region was generated during the off-state of output of the treatment tool 110.

In the modified examples described above, similarly to the first embodiment, in a case where a change in fluorescent regions has been generated in a fluorescence image, an output state of a treatment tool at a time of imaging of the fluorescence image with the generated change is determined, and in a case where the output was off, the newly generated fluorescent region is set as a thermal denaturation region that has been generated during the off-state of output of the energy device and a notification is made to an operating surgeon. The modified example enables the operating surgeon to know about any thermal denaturation that has been generated during the off-state of output of the treatment tool.

Furthermore, in this modified example, even if the time was when the output of the treatment tool 110 was off, if the time is within a predetermined time period from a time at which the treatment tool 110 was switched off, the changed fluorescent region is not set as a new thermal denaturation region and any region enlarged by remaining heat immediately after treatment is excluded from any new thermal denaturation region generated after the output is turned off. The modified example enables the operating surgeon to check only any thermal denaturation region that the operating surgeon does not know of and to efficiently perform treatment because any thermal denaturation region believed to be known by the operating surgeon is excluded from setting of any thermal denaturation region generated after switch-off of the output and any thermal denaturation region after elapse of the predetermined time period is set as a thermal denaturation region to be notified of.

In the example described above with respect to the modified example, any new fluorescent region after elapse of the predetermined time period from the time, at which the treatment tool 110 was turned off, becomes a target to be subjected to the determination for any thermal denaturation region generated when the output was off, but a target to be subjected to the determination may be set according to the enlargement ratio of the fluorescent region instead of the elapsed time period. In that case, on the basis of sizes (spread) of fluorescent regions enlarged by remaining heat, a threshold of the enlargement ratio of a region, or a threshold of a change (difference) in distance from a barycenter position to an outer edge of the region is set, and whether or not the region is to be subjected to the determination is determined on the basis of the threshold.

### Second Embodiment

A second embodiment will be described next. An endoscope system including a rigid scope has been described above with respect to the first embodiment but an endoscope system including a flexible endoscope will be described with respect to this second embodiment. The endoscope system according to the second embodiment will be described hereinafter. For the second embodiment, the same reference sign will be assigned to any component that is that same as that of the endoscope system 1 according to the first embodiment described above and detailed description thereof will be omitted.

### Configuration of Endoscope System

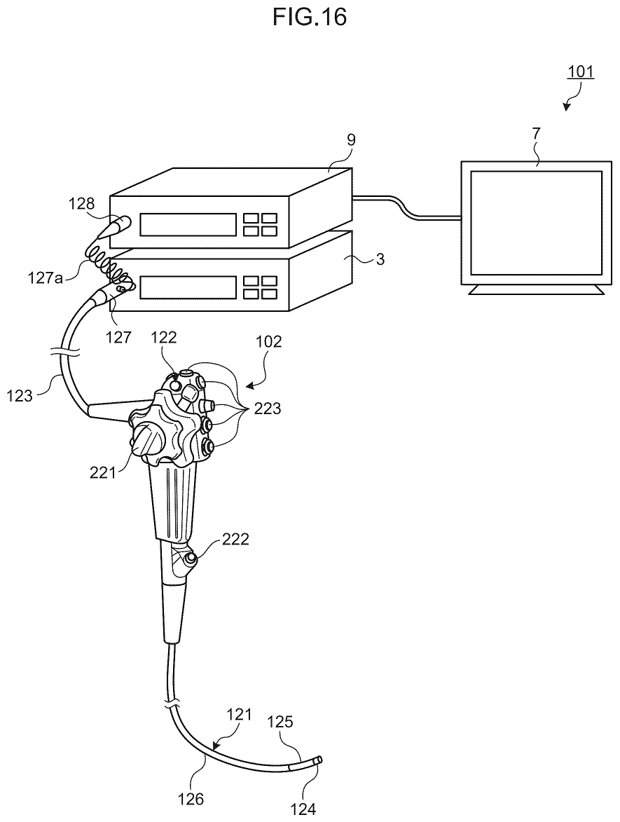

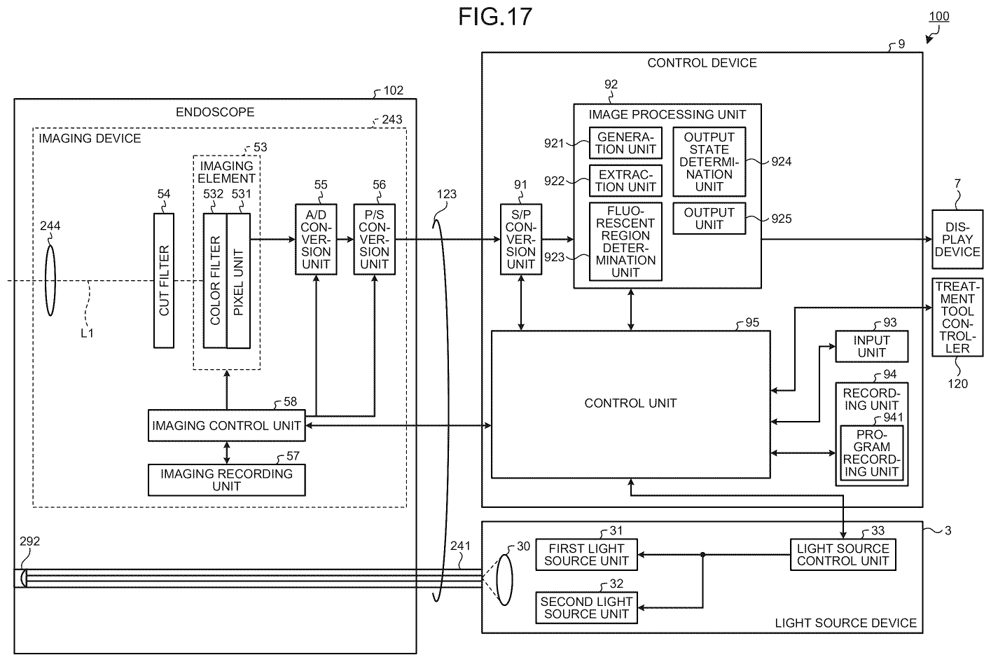

FIG. 16 is a diagram illustrating a schematic configuration of an endoscope system according to a second embodiment. FIG. 17 is a block diagram illustrating a functional configuration of main parts of the endoscope system according to the second embodiment.

In this endoscope system 101, the interior of the body of a subject, such as a patient, is imaged by insertion in the subject and a display image based on image data resulting from this imaging is displayed by the display device 7. By observing the display image displayed by the display device 7, an operating surgeon, such as a medical doctor, examines the presence or absence, and/or the state of each of abnormal regions where bleeding regions, tumor regions, and abnormal regions, which are regions to be examined, have been imaged. Furthermore, an operating surgeon, such as a medical doctor, inserts a treatment tool, such as an energy device, in the body of the subject via a treatment tool channel of an endoscope to perform treatment of the subject. The endoscope system 101 includes an endoscope 102, in addition to the above described light source device 3, display device 7, and control device 9.

### Configuration of Endoscope

The following description is on a configuration of the endoscope 102. By imaging the interior of the body of a subject, the endoscope 102 generates image data and outputs the image data generated, to the control device 9. The endoscope 102 includes an operating unit 122 and a universal cord 123.

An insertion unit 121 has an elongated shape having flexibility. The insertion unit 121 has a distal end portion 124 having a later described built-in imaging device, a bending portion 125 that is freely bendable and includes plural bending pieces, and a flexible tube portion 126 connected to a proximal end of the bending portion 125 and having flexibility and a long shape.

The distal end portion 124 is formed using, for example, glass fiber. The distal end portion 124 has a light guide 241 forming a light guide path for light supplied from the light source device 3, an illumination lens 242 provided at a distal end of the light guide 241, and an imaging device 243.

The imaging device 243 includes an optical system 244 for condensing light, and the above described imaging element 53, cut filter 54, A/D conversion unit 55, P/S conversion unit 56, imaging recording unit 57, and imaging control unit 58 according to the first embodiment.

The universal cord 123 has therein at least the light guide 241 and a bundled cable including one or plural cables bundled together. The bundled cable includes signal lines for transmitting and receiving signals between: the endoscope 102 and light source device 3; and the control device 9, the signal lines including: a signal line for transmitting and receiving set data; a signal line for transmitting and receiving captured images (image data), and a signal line for transmitting and receiving a driving timing signal for driving the imaging element 53. The universal cord 123 has a connector unit 127 attachable to and detachable from the light source device 3. A coil cable 127a having a coil shape is provided to extend from the connector unit 127 and the connector unit 127 has a connector 128 attachable to and detachable from the control device 9, the connector 128 being at an extended end of the coil cable 127a.

The endoscope system 101 configured as described above executes a process similar to that by the endoscope system 1 according to the first embodiment described above.

In the second embodiment described above, similarly to the first embodiment, in a case where a change in fluorescent regions has been generated in a fluorescence image, an output state of a treatment tool at a time of imaging of the fluorescence image with the generated change is determined, and in a case where the output was off, the newly generated fluorescent region is set as a thermal denaturation region generated when the output of the energy device was off and a notification is made to an operating surgeon. The second embodiment enables the operating surgeon to know about any thermal denaturation that has been generated during the off-state of output of the treatment tool.

### Third Embodiment

A third embodiment will be described next. Application to endoscope systems has been described with respect to the first and second embodiments, but application to a surgical microscope system will be described with respect to the third embodiment. For the third embodiment, the same reference sign will be assigned to any component that is that same as that of the endoscope system 1 according to the first embodiment described above and detailed description thereof will be omitted.

### Configuration of Surgical Microscope System

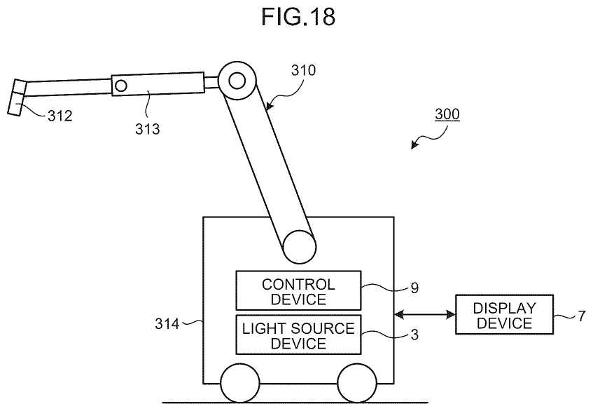

FIG. 18 is a diagram illustrating a schematic configuration of the surgical microscope system according to the third embodiment. A surgical microscope system 300 includes a microscope device 310 that is a medical imaging device that acquires, by imaging, an image for observation of a subject, and a display device 7. The display device 7 and the microscope device 310 may be configured to be integrated with each other.

The microscope device 310 has: a microscope unit 312 that enlarges and images a microscopic region of a subject; a support unit 313 connected to a proximal end of the microscope unit 312 and including an arm that rotatably supports the microscope unit 312; and a base unit 314 that rotatably holds a proximal end portion of the support unit 313 and is capable of moving on a floor surface. The base unit 314 has a light source device 3 that generates white light, first narrowband light, and second narrowband light that are to be emitted to the subject from the microscope device 310, and a control device 9 that controls operation of the surgical microscope system 300. Each of the light source device 3 and the control device 9 has at least a configuration similar to that of the first embodiment described above. Specifically, the light source device 3 includes a condenser lens 30, a first light source unit 31, a second light source unit 32, and a light source control unit 33. The control device 9 includes an S/P conversion unit 91, an image processing unit 92, an input unit 93, a recording unit 94, and a control unit 95. The base unit 314 may be configured to support the support unit 313 by being fixed to a ceiling or a wall surface, for example, instead of being movably provided on the floor surface.

The microscope unit 312 has, for example, a cylindrical shape, and the above described medical imaging device therein. Specifically, the medical imaging device has a configuration similar to that of the endoscope camera head 5 according to the first embodiment described above. For example, the microscope unit 312 includes an optical system 51, a drive unit 52, an imaging element 53, a cut filter 54, an A/D conversion unit 55, a P/S conversion unit 56, an imaging recording unit 57, and an imaging control unit 58. Switches to receive input of operation instructions for the microscope device 310 are provided on a side surface of the microscope unit 312. A cover glass (not illustrated in the drawings) to protect what is inside the microscope unit 312 has been provided in the plane of the opening at a lower end of the microscope unit 312.

A user, such as an operating surgeon, moves the microscope unit 312, performs zoom operation, and switches illumination light, in the surgical microscope system 300 configured as described above, while operating the various switches in a state of holding the microscope unit 312. The microscope unit 312 preferably has an elongated shape extending in an observation direction to allow the user to easily hold the microscope unit 312 and change the viewing direction. The microscope unit 312 may thus have a shape other than a cylindrical shape and may have, for example, a polygonal columnar shape.

In the surgical microscope system 300 according to the third embodiment described above, similarly to the first embodiment, in a case where a change in fluorescent regions has been generated in a fluorescence image, an output state of a treatment tool at a time of imaging of the fluorescence image with the generated change is determined, and in a case where the output was off, the newly generated fluorescent region is set as a thermal denaturation region generated when the output of the energy device was off and a notification is made to an operating surgeon. The third embodiment enables the operating surgeon to know about any thermal denaturation that has been generated during the off-state of output of the treatment tool.

### OTHER EMBODIMENTS

Various embodiments may be formed by combination of plural components as appropriate, the plural components having been disclosed above with respect to the endoscope systems according to the first and second embodiments and the surgical microscope system according to the third embodiment. For example, some of all of the components of the above described endoscope system or surgical microscope system according to an embodiment of the present disclosure may be eliminated. Furthermore, the components described above with respect to the endoscope system or surgical microscope system according to an embodiment of the present disclosure may be combined together as appropriate. The embodiments may be applied to any process based on fluorescence generated by a substance produced by cauterization, for example.

Examples of the process premised on the fact that the first and second fluorescence images are images of the same angle of view have been described above with respect to the embodiments and modified example, but in a case where images having different angles of view and the same subject captured in part thereof are used, fluorescent regions (thermal denaturation regions) are associated with each other using a publicly know method, such as pattern matching, and detection of a change in the fluorescent regions and a process of setting a thermal denaturation region generated after switch-off of the output are then executed.

Furthermore, any “unit” described above for the endoscope system or surgical microscope system according to an embodiment of the present disclosure may be read as a “means” or “circuit”. For example, a control unit may be read as a control means or a control circuit.

In the description of the flowcharts in this specification, the order of the processes at the steps has been clearly stated using expressions, such as “firstly”, “thereafter”, and “subsequently”, but the order of the processes required for implementation of the embodiments is not to be uniquely determined by these expressions. That is, the order of the processes in the flowcharts described in this specification may be modified so long as no contradiction arises from that modification.

Programs to be executed by the devices according to the first to third embodiments may be provided by being recorded as file data in an installable format or executable format in a computer-readable recording medium, such as a CD-ROM, a flexible disk (FD), a CD-R, a digital versatile disk (DVD), a USB medium, or a flash memory.

The programs to be executed by the devices according to the first to third embodiments may be configured to be stored on a computer connected to a network, such as the Internet, and to be provided by being downloaded via the network. The programs to be executed by the devices according to the first to third embodiments may be provided or distributed via a network, such as the Internet.

An example where the light source device 3 is provided separately from the control device 9 has been described with respect to the first and second embodiments, but the light source device 3 and the control device 9 may be configured to be integrated with each other. Furthermore, an example where the light source device 3 is integrated with the control device 9 has been described with respect to the third embodiment, but the light source device 3 and the control device 9 may be configured as separate devices.

Some of embodiments of the present application have been described above in detail on the basis of the drawings but these are just examples, and the present invention may be implemented in other modes, to which various modifications and improvements have been made on the basis of the embodiments disclosed herein and knowledge of those skilled in the art.

As described above, a medical device, a medical system, a medical device operation method, and a medical device operation program, according to the disclosure, are useful for letting an operating surgeon know about thermal denaturation that occurred when output of a treatment tool was off.

An effect achieved according to the present disclosure is to enable an operating surgeon to get a grasp of thermal denaturation that has occurred during an off-state of output of a treatment tool.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device, comprising:
a processor comprising hardware, the processor being configured to
generate a fluorescence image based on fluorescence generated by excitation light that excites a substance produced by cauterization using an energy device,
determine, based on output information on the energy device and on the fluorescence image, an off-time generated fluorescent region that has been generated during an off-state of output of the energy device, and
when it is determined to be the off-time generated fluorescent region, execute a notification process of notifying that a fluorescent region has been generated during the off-state of output of the energy device.

2. The medical device according to claim 1, wherein the processor is further configured to
generate a first fluorescence image, and a second fluorescence image imaged later than the first fluorescence image,
determine, based on the first fluorescence image and the second fluorescence image, whether or not there is a new fluorescent region present only in the second fluorescence image, and
when there is the new fluorescent region, determine, based on the output information on the energy device, whether or not the new fluorescent region is the off-time generated fluorescent region.

3. The medical device according to claim 2, wherein
the first fluorescence image is an image that has been imaged during an on-state of output of the energy device, and
the second fluorescence image is an image that has been imaged during the off-state of output of the energy device.

4. The medical device according to claim 2, wherein the processor is further configured to
extract a first fluorescent region from the first fluorescence image,
extract a second fluorescent region from the second fluorescence image, and
determine whether or not there is the new fluorescent region by comparing the extracted first fluorescent region with the extracted second fluorescent region.

5. The medical device according to claim 1, wherein the fluorescence is light generated by excitation of the substance.

6. The medical device according to claim 5, wherein the substance is an advanced glycation end-product produced by thermal denaturation.

7. The medical device according to claim 1, wherein the processor is further configured to determine whether or not there is the off-time generated fluorescent region in a fluorescent region of a second fluorescence image imaged after elapse of a preset time period from a time when the energy device was switched off.

8. The medical device according to claim 1, wherein the processor is further configured to generate a display image having the off-time generated fluorescent region and a fluorescent region other than the off-time generated fluorescent region that are displayed in modes different from each other.

9. The medical device according to claim 8, wherein the processor is further configured to generate the display image having the off-time generated fluorescent region and the fluorescent region other than the off-time generated fluorescent region that are displayed in the modes different from each other on the second fluorescence image.

10. The medical device according to claim 8, wherein the processor is further configured to
generate a white light image based on reflected light and returned light from a biological tissue upon irradiation of the biological tissue with white light, and
generate the display image having the off-time generated fluorescent region and the fluorescent region other than the off-time generated fluorescent region that are displayed in the modes different from each other on the white light image.

11. A medical device, comprising:
a processor comprising hardware, the processor being configured to
determine, based on a fluorescence image based on fluorescence generated by excitation light that excites a substance produced by cauterization using an energy device and output information on the energy device, an off-time generated fluorescent region that has been generated during an off-state of output of the energy device, and
when it is determined to be the off-time generated fluorescent region, execute a notification process of notifying that a fluorescent region has been generated during the off-state of output of the energy device.

12. A medical system, comprising:
an imaging device configured to image a subject;
a light source configured to emit excitation light that excites a substance produced by a heat treatment on a biological tissue; and
a control device that the imaging device is attachable to and detachable from, the control device comprising a processor and being capable of communicating with a controller that controls an energy device configured to cauterize a treatment target, the processor being configured to
generate a fluorescence image based on fluorescence generated by the excitation light that excites the substance produced by cauterization using the energy device,
determine, based on output information on the energy device and on the fluorescence image, an off-time generated fluorescent region that has been generated during an off-state of output of the energy device, and
when it is determined to be the off-time generated fluorescent region, execute a notification process of notifying that a fluorescent region has been generated during the off-state of output of the energy device.

13. A medical device operation method executed by a medical device, the method comprising:
generating a fluorescence image based on fluorescence generated by excitation light that excites a substance produced by cauterization using an energy device,
determining, based on output information on the energy device and on the fluorescence image, an off-time generated fluorescent region that has been generated during an off-state of output of the energy device, and
when it is determined to be the off-time generated fluorescent region, notifying that a fluorescent region has been generated during the off-state of output of the energy device.

14. A non-transitory computer-readable recording medium with an executable program stored thereon, the program causing a medical device to execute:
generating a fluorescence image based on fluorescence generated by excitation light that excites a substance produced by cauterization using an energy device,
determining, based on output information on the energy device and on the fluorescence image, an off-time generated fluorescent region that has been generated during an off-state of output of the energy device, and
when it is determined to be the off-time generated fluorescent region, notifying that a fluorescent region has been generated during the off-state of output of the energy device.
